(19) United States
(12) Patent Application Publication
(10) Pub. No.: US 2013/0237822 A1
(43) Pub. Date: Sep. 12, 2013

(54) COMBINED RADIOTHERAPY ULTRASOUND DEVICE

(71) Applicants: Patrick Gross, Langensendelbach (DE); Björn Heismann, Erlangen (DE)

(72) Inventors: Patrick Gross, Langensendelbach (DE); Björn Heismann, Erlangen (DE)

(21) Appl. No.: 13/414,527

(22) Filed: Mar. 7, 2012

(51) Int. Cl. A61B 8/13; A61N 5/00

(52) U.S. Cl. 600/439

(57) ABSTRACT

In order to minimize radiation directed to a region outside of a target volume of a patient during a radiation therapy and minimize the cost of an image-guided particle therapy system, the particle therapy system includes an ultrasound device having at least one ultrasound transducer positioned abutting or adjacent to an external surface of the patient. The ultrasound device and thus the ultrasound transducer are positioned outside of a beam path of a particle beam between a radiotherapy device generating the particle beam, and the target volume. The ultrasound device is operable to generate data representing the target volume and/or a region outside of the target region while the radiotherapy device directs the particle beam to the target volume. The particle therapy system includes a processor operable to control the radiotherapy device based on a comparison between the generated data representing the target volume and a predetermined treatment plan.

### FIELD

The present embodiments relate to a combined radiotherapy and ultrasound device.

### BACKGROUND

In radiation therapy, high energy particle beams are used to damage and ultimately destroy cancerous tissue in a human body. Breast, brain, abdomen, lung, or prostate tumors, for example, may be targets. Due to organ motion (e.g., motion of the lungs during breathing by the patient), the radiation therapy may be image guided using an imaging modality.

The imaging modality may be a computed tomography (CT) system. A radiotherapy device (e.g., a linear accelerator (LINAC)) may be mounted on a gantry of the CT system (e.g., a combined LINAC-CT system). The combined LINAC-CT system, however, increases the radiation dose delivered to the patient due to the additional radiation delivered to the patient by the CT system.

The imaging modality may also be a magnetic resonance imaging (MRI) system. A radiotherapy device (e.g., a LINAC) may be vertically positioned in an almost field-free region of a split magnet MR system (e.g., a combined LINAC-MRI system). The combination of a strong magnetic field and an electron beam within the combined LINAC-MRI system, for example, poses technical challenges, and the cost of the combined LINAC-MRI system may be high.

### SUMMARY

In order to minimize radiation directed to a region outside of a target volume of a patient during a radiation therapy and minimize the cost of an image-guided particle therapy system, the particle therapy system is combined with ultrasound imaging. The combined system includes an ultrasound device having at least one ultrasound transducer positioned abutting or adjacent to an external surface of the patient. The transducer may be within the patient, such as in a catheter. The ultrasound device and thus the ultrasound transducer are positioned outside of a beam path of a particle beam between a radiotherapy device generating the particle beam and the target volume. The ultrasound device is operable to generate data representing the target volume and/or a region outside of the target region while the radiotherapy device directs the particle beam to the target volume. The particle therapy system includes a processor operable to control the radiotherapy device based on a comparison between the generated data representing the target volume and a predetermined treatment plan.

In a first aspect, a system for irradiating a target volume with a radiation or particle beam includes a radiotherapy device operable to irradiate the target volume with the radiation or particle beam. The system also includes an ultrasound device including an ultrasound transducer. The ultrasound device is operable to generate ultrasound data representing at least part of the target volume. The ultrasound transducer is positioned adjacent to the target volume. The system includes a memory configured to store a treatment plan for irradiating the target volume with the radiation or particle beam. The system also includes a processor operatively connected to the radiotherapy device, the ultrasound transducer, and the memory. The processor is configured to generate image data corresponding to at least the part of the target volume based on the generated ultrasound data. The processor is also configured to control the radiotherapy device based on the generated image data and the stored treatment plan.

In a second aspect, a method of correcting for target volume motion for an irradiation of a target volume of a patient with a radiation or particle beam includes positioning a support structure on or adjacent to an external surface of the patient. The support structure supports an ultrasound transducer. The ultrasound transducer is outside of a beam path of the radiation or particle beam from the radiotherapy device to the target volume. The method also includes generating image data representing at least part of the target volume using the ultrasound transducer. The method includes irradiating the target volume with the radiotherapy device and controlling the irradiation of the target volume based on the generated image data.

In a third aspect, a non-transitory computer-readable storage medium stores instructions executable by one or more processors to correct for motion of a target volume during a radiation therapy with a radiation or particle beam. The instructions include receiving data representing at least part of the treatment volume from an ultrasound transducer. The ultrasound transducer is outside of a beam path of the radiation or particle beam from the radiotherapy device to the target volume. The instructions also include controlling irradiation of the target volume. The controlling includes turning the radiation or particle beam on and off, moving the radiation or particle beam, or changing a shape of the radiation or particle beam.

### BRIEF DESCRIPTION OF THE DRAWINGS

FIG. 1 shows one embodiment of an image-guided system for irradiating a target volume with a particle beam;

FIG. 2 shows one embodiment of on an ultrasound device;

FIG. 3 shows another embodiment of an ultrasound device;

FIG. 4 shows one embodiment of an ultrasound system including the ultrasound device of FIG. 2 or FIG. 3;

FIG. 5 shows a flowchart of one embodiment of a method of correcting for target volume motion for an irradiation of a target volume.

### DETAILED DESCRIPTION OF THE DRAWINGS

In radiation therapy, high-energy quanta are used to damage and ultimately destroy cancerous tissue in a human body. Breast, brain, abdomen, lung or prostate tumors may be targets. Due to organ motion, radiotherapy is combined with a diagnostic ultrasound system. This allows the tumor to be tracked on-line, while minimizing the radiation delivered to regions of a patient outside the tumor.

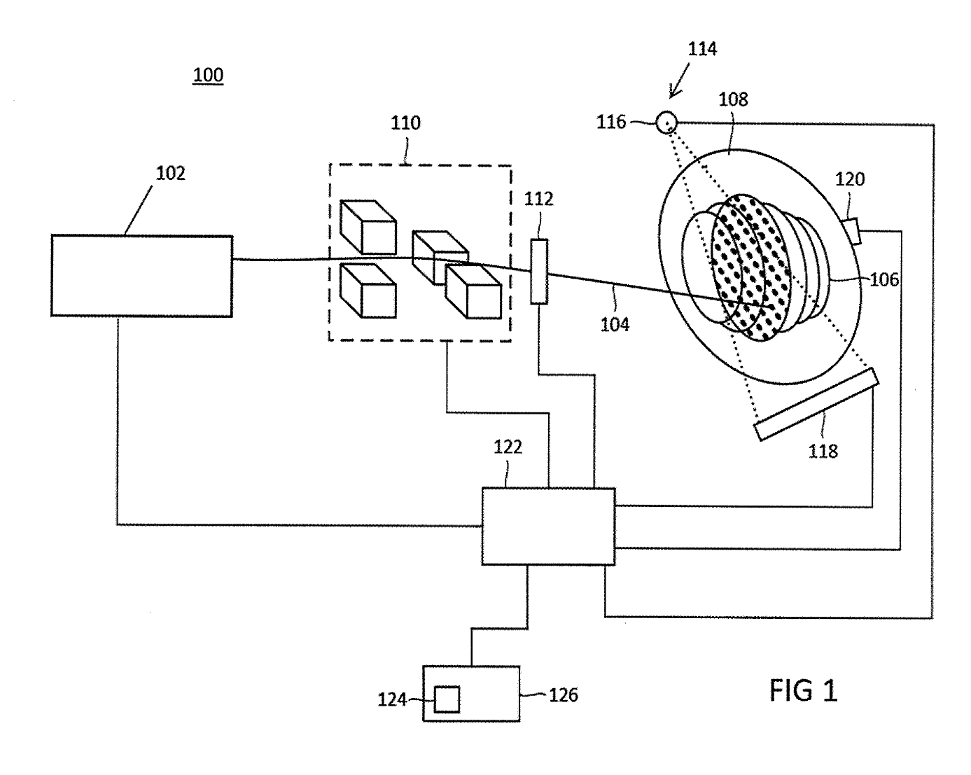

FIG. 1 shows one embodiment of an image-guided particle therapy system 100 (e.g., a particle therapy system). The particle therapy system 100 includes a radiotherapy device 102 such as, for example, a linear accelerator (LINAC) that provides a particle beam 104 with energy for an irradiation. The LINAC 102 may accelerate electrons to an energy between, for example 4 and 25 MeV. The particle beam 104 may be used to irradiate a target volume 106 located on a table (e.g., a patient table or a patient bed). In one embodiment, the LINAC 102 may include other components such as, for example, a syncrotron. Other radiotherapy devices such as, for example, electron or ion beam sources, Cobalt-based radiation therapy or radiation surgery systems, and particle therapy systems may be used. The particle beam 104 may include charged particles such as, for example, electrons, protons, pions, helium ions, carbon ions, or ions of other elements.

The target volume 106 may, for example, be tumor-diseased tissue of the patient. In one embodiment, the particle therapy system 100 may also be used, for example, to irradiate a non-living body such as, for example, a water phantom or other type of phantom, or cell cultures for research or maintenance purposes. Objects that form the target volume 106 may be moving bodies (e.g., a tumor within a lung of the patient that moves due to breathing) or only possibly moving bodies (e.g., a tumor in the arm, leg or head that may move due to patient voluntary motion). The target volume 106 may be non-visibly located inside a target object 108 (e.g., the patient).

A raster scanning method, in which the particle beam 104 is guided from target point to target point without being turned off when a transition is made from one target point to the next, may be used as the scanning method. The particle beam 104 provided by the LINAC 102 may be influenced in a lateral deflection with the aid of scanning magnets 110. The particle beam 104 may, for example, be deflected in a direction that is perpendicular to a beam trajectory direction (e.g., the x- and y-directions). In other embodiments, other scanning methods may be used. For example, passive beam application may be used.

In one embodiment, the LINAC 102 may irradiate the target volume 106 while at least part of the LINAC 102 rotates about the target volume 106. For example, the LINAC 102 may include a gantry (not shown) operable to rotate at least the part of the LINAC 102 about the target volume 106 before, during, and/or after the irradiation of the target volume 106. Alternatively, the LINAC 102 may remain stationary relative to the target volume 106 during the irradiation of the target volume 106.

The particle therapy system 100 may also include a beam shaping device 112. The beam shaping device 112 may, for example, be a multileaf collimator. The multileaf collimator 112 may be used to approximately match a shape of the particle beam 104 to a shape of the target volume 106. The multileaf collimator 112 may include a plurality of individually movable leaves, and the higher the number of individually movable leaves, the more precisely the multileaf collimator 112 may shape the particle beam 104 to match the shape of the target volume 106. The multileaf collimator 112 may, for example, include 50-120 leaves. The multileaf collimator 112 may be made of any number of materials including, for example, Tungsten. The scanning magnets 110 and the beam shaping device 112 may be in the same housing as the LINAC 102 or may be components separate from the LINAC 102.

The particle therapy system 100 may also include an imaging device 114. In the embodiment shown in FIG. 1, the imaging device 114 is an X-ray device that includes a radiation source 116 and a radiation detector 118. The radiation detector 118 may generate two-dimensional (2D) datasets representing the target volume 106. The 2D datasets may be further processed to generate three-dimensional (3D) datasets (e.g., volumetric datasets). The 2D datasets may be obtained contemporaneously with the planning of a medical treatment procedure (e.g., to irradiate and destroy cancerous tissue within the target volume 106). For example, the imaging device 114 may be used to create a patient model that may be used in the planning of the medical treatment procedure (e.g., part of a treatment plan). In other embodiments, the imaging device 114 may be a computed tomography (CT) device, a positron emission tomography (PET) device, an angiography device, a fluoroscopy device, or an ultrasound device. The imaging device 114 may be in the same or different room as the LINAC 102. The imaging device 114 may be in the same or different facility (e.g., hospital) as the LINAC 102. In one embodiment, the particle therapy system 100 does not include the imaging device 114; instead, the 2D datasets are received from another facility (e.g., a remote facility) that generated the 2D datasets at an earlier time.

The particle therapy system 100 also includes an ultrasound device 120. During the radiation therapy for the tumor (e.g., the target volume 106) in the lung of the patient, for example, the lung moves as the patient breathes. In order to track the movement of the target volume 106 during the radiation therapy, the ultrasound device 120 generates data (e.g., ultrasound data) representing the target volume 106 and a region outside of the target volume 106 during the radiation therapy. The ultrasound device 120 may be operated at a frame rate of, for example, 10 or more frames per second to provide a temporal resolution sufficient to stabilize the particle beam 104 in the target object 108. Other frame rates, however, may be used.

The ultrasound device 120 may be positioned in physical contact with or adjacent to the target object 108. “Adjacent” may include arrangements where there are one or more intervening materials (e.g., a gel pad or a water bath) between the ultrasound device 120 and the target object 108. In one embodiment, the ultrasound device 120 may be positioned in physical contact with an external surface of the target object 108. The ultrasound device 120 may be positioned on the external surface of the target object 108 such that the ultrasound device 120 is outside of a beam path of the particle beam 104 between the LINAC 102 and the target volume 106 (e.g., absorbing materials are kept outside of the beam path of the particle beam 104 between the LINAC 102 and the target volume 106).

In one embodiment, the ultrasound device 120 also acts as the imaging device 114. For example, in addition to tracking the movement of the target volume 106 during the radiation therapy, the ultrasound device 120 may also be used to create the patient model that may be used in the planning of the medical treatment procedure (e.g., part of the treatment plan).

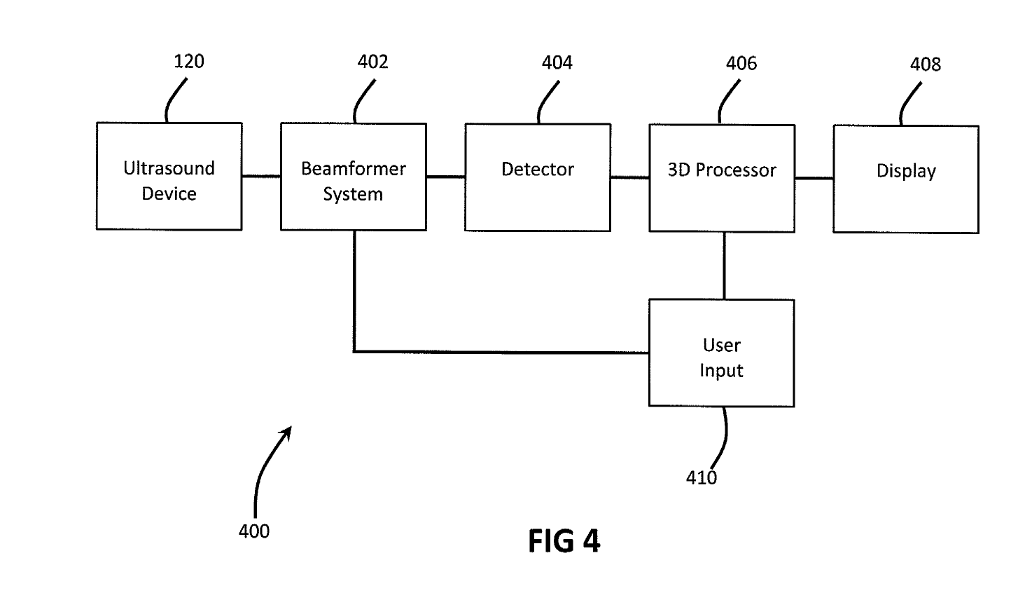

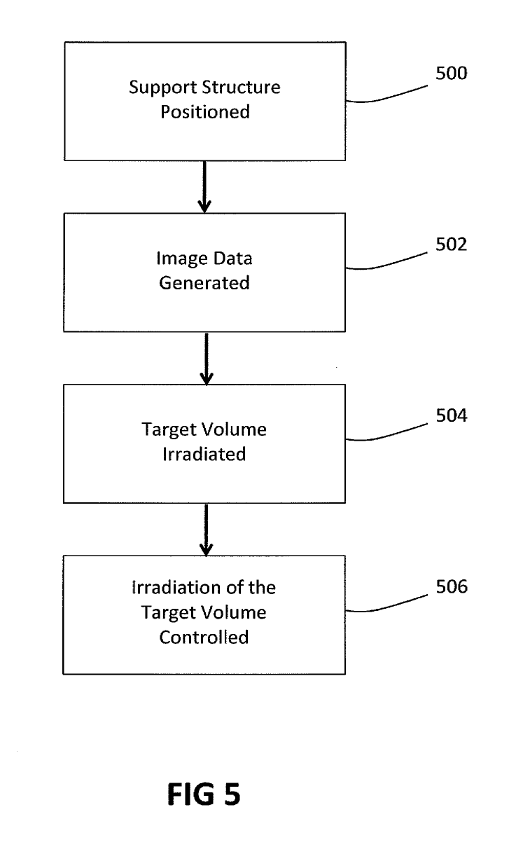

The ultrasound device 120 includes at least one transducer (e.g., an array of transducers, such as shown in FIGS. 3 and 4). The ultrasound device 120 may generate an ultrasound beam, for example. The transducer is a multidimensional transducer array, a one-dimensional transducer array, a wobbler transducer or another transducer operable to scan mechanically and/or electronically in a plane or volume. For example, a multidimensional transducer array electronically scans along scan lines positioned at different locations within the target volume 106. As another example, a one-dimensional transducer array is rotated by a mechanism within a plane along a face of the one-dimensional transducer array or an axis spaced away from the one-dimensional transducer array for scanning a plurality planes within the target volume 106. As yet another embodiment, a wobbler transducer array is operable to scan a plurality of planes spaced in different positions within the target volume 106. In one embodiment, some or all of the plurality of transducers are capacitive micromachined ultrasonic transducers (CMUTs), piezoelectric transducers, composite-based transducers, or a combination thereof. In another embodiment, at least one transducer of the plurality of transducers is a high intensity focused ultrasound (HIFU) transducer used to ablate the target volume 106, thus aiding the LINAC 102 in treating the target volume 106. The scan is of any format such as, for example, a sector scan along a plurality of frames in two dimensions and a linear or sector scan along a third dimension. Linear or vector scans may alternatively be used in any of the various dimensions.

Electronics (e.g., the at least one transducer) of the ultrasound device 120 are shielded from radiation generated during the medical treatment procedure. For example, one or more layers of one or more high impedance materials and/or compounds may be used to shield the electronics of the ultrasound device 120. Any number of high impedance materials including, for example, tungsten and lead, may be used to shield the electronics of the ultrasound device 120 from ionizing effects.

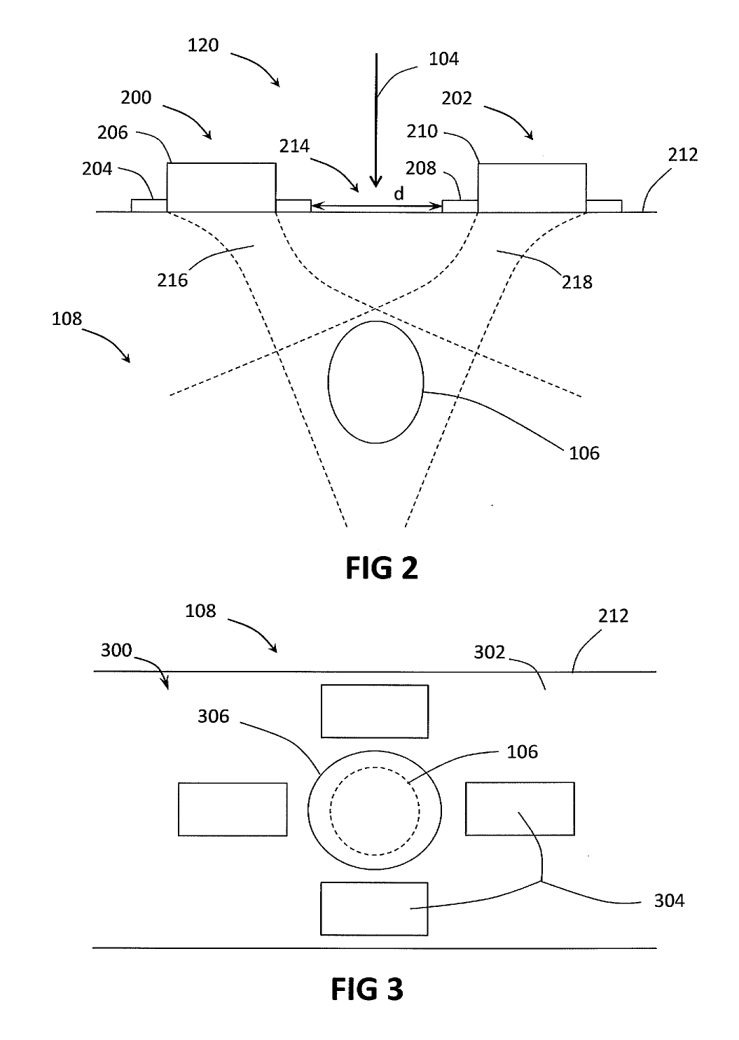

FIG. 2 shows a cross-sectional view of one embodiment of the ultrasound device 120. The ultrasound device 120 includes a first ultrasound assembly 200 and a second ultrasound assembly 202. The first ultrasound assembly 200 includes a first support structure 204 and at least one first transducer 206. The second ultrasound assembly 202 includes a second support structure 208 and at last one second transducer 210. The first support structure 204 and the second support structure 208 may be used to position the at least one first transducer 206 and the at least one second transducer 210, such that the at least one first transducer 206 and/or the at least one second transducer 210 are in physical contact with, abut, or are adjacent to an external surface 212 of the target object 108 (e.g., an external surface of the chest of the patient). The first ultrasound assembly 200 and/or the second ultrasound assembly 202 may be belts, blankets or cuffs. The ultrasound device 120 may include more or fewer ultrasound assemblies.

The at least one first transducer 206 (e.g., a first transducer) and the at least one second transducer 208 (e.g., a second transducer) may be attached to the first support structure 204 and the second support structure 208, respectively, in any number of ways. For example, the first transducer 206 and/or the second transducer 210 may be press fit into part of the first support structure 204 and/or part of the second support structure 208, respectively. Additionally or alternatively, the first transducer 206 and/or the second transducer 210 may be attached to the first support structure 204 and/or the second support structure 208, respectively, with one or more fastening devices. The one or more fastening devices may include, for example, adhesives, nut/bolt combinations, tabs, snap fit detention and extensions, combinations thereof, or any other now known or later discovered fastening device. In one embodiment, part of the first support structure 204 and/or part of the second support structure 208 may surround the first transducer 206 and/or the second transducer 210, respectively. The first support structure 204 and/or the second support structure 208 may be made of any number of flexible materials including, for example, polyethylene or silicon rubber. The first support structure 204 and/or the second support structure 208 may be any number of lengths and widths. In one embodiment, the first support structure 204 and/or the second support structure 208 are sized to wrap around the circumference of a portion of the target object 108 (e.g., the circumference of the chest of the patient). The first support structure 204 and the second support structure 208 may include one or more tightening mechanisms operable to tightly fix the first ultrasound assembly 200 and/or the second ultrasound assembly 202 relative to the external surface 212 of the target object 108. The one or more tightening mechanisms may be buckles, buttons, Velcro, adhesives, combinations thereof, or any other now known or later discovered tightening mechanisms.

In one embodiment, the first ultrasound assembly 200 and/or the second ultrasound assembly 202 may not include any support structures, and the first transducer 206 and/or the second transducer 210 may be adhered directly to the external surface 212 of the target object 108. In another embodiment, the first transducer 206 and/or the second transducer 210 may be embedded in the patient bed of the LINAC 102. In yet other embodiments, some or all of a plurality of first transducers 206 and/or a plurality of second transducers 210 may be integrated into a mask molded to the face or another body part of the patient and operable to fix the head or the other body part of the patient relative to the LINAC 102. The mask may be thermoplastic devices, vacuum cushions, or other now known or later developed devices for fixing the patient relative to the LINAC 102.

The first ultrasound assembly 200 and the second ultrasound assembly 202 are positioned on the external surface 212 of the target object 108 (e.g., the patient) relative to the target volume 106, such that the first ultrasound assembly 200 and the second ultrasound assembly 202 are out of the beam path of the particle beam 104. The first ultrasound assembly 200 and the second ultrasound assembly 202 are separated by a distance d. The distance d may be determined by the size of a field of view of the LINAC 102. In one embodiment, a target region 214 above the target volume 106 is kept free of absorbing materials. Thus, a boundary free radiation beam path may be provided.

In order to image the target volume 106 and/or the region outside of the target volume 106, an ultrasound beam 216 of the first transducer 206 and an ultrasound beam 218 of the second transducer 210 are, for example, directed towards a center of an exposure region (e.g., the target volume 106) of the particle beam 104. Alternatively or additionally, the ultrasound beam 216 and the ultrasound beam 218 sweep tissue regions adjacent to and including or excluding the exposure region. Any scan format may be used. Multiple ultrasound transducers may increase volume coverage and temporal resolution.

In one embodiment, the gantry of the LINAC 102 rotates while the LINAC 102 delivers radiation to the target volume 106. The first ultrasound assembly 200 and the second ultrasound assembly 202 may each include a plurality of transducers positioned along the length of the first support structure 204 and the second support structure 208, respectively. The plurality of transducers may be positioned on each of the first support structure 204 and the second support structure 208, such that the plurality of transducers form rings around the target object 108. The plurality of transducers may include any number of transducers. For example, the number of transducers along the lengths of the first support structure 204 and the second support structure 208 may be determined such that the target volume 106 and/or the region outside of the target volume 106 may be imaged along various angles of incidence of the particle beam 104. The ultrasound device 120 (e.g., the first ultrasound assembly 200 and the second ultrasound assembly 202) blocks a strip around the patient, but the therapy beam may otherwise be moved to interact with the target from various directions.

FIG. 3 shows a top view of another embodiment of the ultrasound device 120. The ultrasound device 120 includes an ultrasound assembly 300. The ultrasound assembly 300 includes a support structure 302 and at least one transducer 304 (e.g., four transducers). The support structure 302 may include an opening 306. The four transducers 304 may be positioned on the support structure 302 such that the four transducers 304 are disposed around (e.g., surround) the opening 306. The ultrasound assembly 300 may be positioned on the external surface 212 of the target object 108, such that an outer edge defining the opening 306 surrounds the target region 214, and the four transducers 304 are disposed around the target region 214. The diameter of the opening 306 and the position of the ultrasound assembly 300 may be determined using data corresponding to the target volume 106 generated by the imaging device 114. The ultrasound assembly 300 is thus out of the beam path of the particle beam 104. The target region 214 above the target volume 106 is kept free of absorbing materials. A boundary free radiation beam path may thus be provided. The support structure 302 may be used to position the four transducers 304, such that the four transducers 304 are in physical contact with, abut, or are adjacent with the external surface 212 (e.g., an external surface of the chest of the patient) of the target object 108 (e.g., the patient). The ultrasound assembly 300 may be a belt, a blanket, or a cuff.

The four transducers 304 may be attached to the support structure 302 in any number of ways. For example, the four transducers 304 may be press fit into part of the support structure 302. Additionally or alternatively, the four transducers 304 may be attached to the support structure 302 with one or more fastening devices. The one or more fastening devices may include, for example, adhesives, nut/bolt combinations, tabs, combinations thereof, or any other now known or later discovered fastening device. In one embodiment, part of the support structure 302 may surround some or all of the four transducers 206. The support structure 302 may be made of any number of flexible materials including, for example, polyethylene or silicon rubber. The support structure 302 may be any number of lengths and widths. In one embodiment, the support structure 302 is sized to wrap around the circumference of a portion of the target object 108 (e.g., the circumference of the chest of the patient). The support structure 302 may include one or more tightening mechanisms operable to tightly fix the ultrasound assembly 300 relative to the external surface 212 of the target object 108. The one or more tightening mechanisms may be buckles, buttons, Velcro, adhesives, combinations thereof, or any other now known or later discovered tightening mechanisms.

In one embodiment, the ultrasound assembly 300 may not include any support structures, and the four transducers 304 may be adhered directly to the external surface 212 of the target object 108. In another embodiment, the four transducers 304 may be embedded in the patient bed of the LINAC 102. In yet other embodiments, the four transducers 304 may be integrated into a mask molded to the face or another body part of the patient and operable to fix the head or the other body part of the patient relative to the LINAC 102. The mask may be thermoplastic devices, vacuum cushions, or other now known or later developed devices for fixing the patient relative to the LINAC 102.

In order to image the target volume 106 and/or the region outside of the target volume 106, ultrasound beams (not shown), for example, of the four transducers 304 are directed towards a center of the exposure region (e.g., the target volume 106) of the particle beam 104. Each of the transducers 304 may be an array of elements allowing electronic steering of scan beams. Alternatively or additionally, the ultrasound beams of the four transducers 304 sweep tissue regions adjacent to the exposure region. Additional ultrasound transducers may increase volume coverage and temporal resolution.

In one embodiment, the gantry of the LINAC 102 remains stationary while the LINAC 102 delivers radiation to the target volume 106 through the opening 306 in the ultrasound assembly 300. The four transducers 304 image the target volume 106 while the LINAC 102 delivers the radiation to the target volume 106.

FIG. 4 shows one embodiment of an ultrasound system 400 for assisting in 3D ultrasound imaging of the target volume 106. The ultrasound system 400 includes, for example, the ultrasound device 120, a beamformer system 402, a detector 404, a 3D rendering processor 406, a display 408, and a user input 410. The ultrasound system 400 may include more or fewer components.

The ultrasound device 120 (e.g., the first ultrasound assembly 200 and the second ultrasound assembly 202) may include components other than the first transducer 206 and the second transducer 210. For example, the other components may include one or more multiplexers, one or more processors, one or more memories, one or more sub-array beamformers, fiber optics for determining relative position of the transducers (e.g., the first transducer 206 and the second transducer 210), one or more accelerometers for measuring the movement of the ultrasound device 120, or combinations thereof. The ultrasound device is in communication with the beamformer system 402 wirelessly or via one or more cables (e.g., a coaxial cable), for example.

The beamformer system 402 is a transmit beamformer, a receive beamformer, a controller for a wobbler array, filters, a position sensor, combinations thereof or other now known or later developed components for scanning in three-dimensions. The beamformer system 402 is operable to generate waveforms and receive electrical echo signals for scanning the target volume 106. The beamformer system 402 controls the beam spacing with electronic and/or mechanical scanning. For example, a wobbler transducer displaces a one-dimensional array to cause different planes within the volume to be scanned electronically in two-dimensions.

The detector 404 is a B-mode detector, a Doppler detector, a video filter, a temporal filter, a spatial filter, a processor, an image processor, combinations thereof or other now known or later developed components for generating image information from the acquired ultrasound data output by the beamformer system 402. In one embodiment, the detector 404 includes a scan converter for scan converting two-dimensional scans in a polar coordinate format within a volume associated with frames of data to a Cartesian coordinate format for a display. In other embodiments, the data is provided for representing the target volume 106 without scan conversion.

The 3D processor 406 is a general processor, a data signal processor, a graphics card, a graphics chip, a personal computer, a motherboard, memories, buffers, scan converters, filters, interpolators, a field programmable gate array, an application specific integrated circuit (ASIC), analog circuits, digital circuits, combinations thereof or any other now known or later developed device for generating three-dimensional or two-dimensional representations from input data in any one or more of various formats. The 3D processor 406 includes software or hardware for rendering a three-dimensional representation of the target volume 106 and/or the region outside of the target volume 106, such as through alpha blending, minimum intensity projection, maximum intensity projection, surface rendering, or other now known or later developed rendering techniques. The 3D processor 406 may have software for generating a two-dimensional image corresponding to any plane through the target volume 106 and/or the region outside of the target volume 106. The software may allow for a three-dimensional rendering bounded by a plane through the target volume 106 or a three-dimensional rendering for a region around the plane. The 3D processor 406 is operable to render an ultrasound image representing the target volume 106 from data acquired by the beamformer system 402. Alternatively or additionally, the 3D processor 406 is operable to identify tissue or features from the data representing the target volume 106.

The display 408 is a monitor, a cathode ray tube (CRT), a liquid crystal display (LCD), a plasma screen, a flat panel projector, or another now known or later developed display device. The display 408 is operable to generate images for a two-dimensional view or a rendered three-dimensional representation. For example, a two-dimensional image representing a three-dimensional volume through rendering is displayed.

The user input 410 is a keyboard, touch screen, mouse, trackball, touchpad, dials, knobs, sliders, buttons, combinations thereof or other now known or later developed user input devices. The user input 410 connects with the beamformer system 402 and the 3D processor 406. Input from the user input 410 controls the acquisition of data and the generation of images. For example, the user manipulates buttons and a track ball or mouse for indicating a viewing direction, a type of rendering, a type of examination, a specific type of image (e.g., an A4C image of the lung), an acoustic window being used, a type of display format, landmarks on an image, combinations thereof or other now known or later developed two-dimensional imaging and/or three-dimensional rendering controls. In one embodiment, the user input 410 is used during real time imaging, such as streaming volumes (e.g., four dimensional imaging for tracking the movement of the target volume 106).

As shown in FIG. 1, the particle therapy system 100 also includes a controller 122 in communication with a memory 126. The controller 122 may be in communication with and controls the operation of the LINAC 102, the scanning magnets 110, and/or the beam shaping device 112. In one embodiment, the controller 122 is in communication with and controls the operation of the imaging device 114 (e.g., the radiation source 116 and the radiation detector 118).

The controller 122 is a general processor, a central processing unit, a control processor, a graphics processor, a digital signal processor, a three-dimensional rendering processor, an image processor, an ASIC, a field-programmable gate array, a digital circuit, an analog circuit, combinations thereof, or another now known or later developed controller. The controller 122 is a single device or multiple devices operating in serial, parallel, or separately. The controller 122 may be a main processor of a computer such as a laptop or desktop computer, or may be a processor for handling some tasks in a larger system. For example, the controller 122 may be the 3D processor 406 of the ultrasound system 400 or a processor of the therapy system 100. The controller 122 is configured by instructions, design, hardware, and/or software to perform the acts discussed herein, such as performing target volume motion correction for an irradiation of the target volume 106.

The memory 126 is a computer readable storage media. The computer readable storage media may include various types of volatile and non-volatile storage media, including but not limited to random access memory, read-only memory, programmable read-only memory, electrically programmable read-only memory, electrically erasable read-only memory, flash memory, magnetic tape or disk, optical media and the like. The memory 126 may be a single device or a combination of devices. The memory may be adjacent to, part of, networked with and/or remote from the controller 122.

The LINAC 102, the scanning magnets 110, and the beam shaping device 112 may be controlled based on the ultrasound data generated by the ultrasound system 400 and a treatment plan 124 stored in the memory 126, such that radiation reaching the region outside of the target volume 106 may be minimized. The treatment plan 124 includes a three-dimensional representation of the target volume 106 generated before conducting the medical treatment procedure using the LINAC 102. The three-dimensional representation of the treatment volume 106 may be generated using the imaging device 114, for example. The treatment plan 124 also includes, for example, a sequence of delivery segments, within which discrete points are described by, for example, a beam shape (i.e., a shape and/or an orientation of the beam shaping device 112), a beam dose, a beam energy, and/or gantry angles defining a range or span of the segment (e.g., an upper limit and a lower limit), within which the radiation dose is to be delivered.

In one embodiment, the treatment plan 124 is for an intensity modulated radiation therapy (IMRT) methodology, where the gantry of the LINAC 102 delivers radiation to the target volume 106 at one or more gantry angles. The IMRT methodology may be a stop-and-shoot IMRT methodology, where the gantry of the LINAC 102 rotates and stops at one or more gantry angles, at which the LINAC 102 delivers radiation to the target volume 106. Alternatively, the LINAC 102 may deliver radiation to the target volume 106 while the gantry of the LINAC 102 is rotating. The LINAC 102 may deliver radiation to the target volume 106 continuously during rotation of the gantry, or may deliver radiation to the target volume 106 in segments (e.g., 15 degrees to 30 degrees and 45 degrees to 60 degrees) of the rotation of the gantry.

The controller 122 may register the imaging device 114 and the ultrasound device 120 with the LINAC 102, and/or may register data generated with the imaging device 114 and the ultrasound device 120 with the LINAC 102. For example, data generated by the imaging device 114 and the ultrasound device 120 may be transformed into a coordinate system of the LINAC 102. In one embodiment, fiducial markers embedded in the patient bed (e.g., of the LINAC 102) may be used to register data generated by the imaging device 114 with the LINAC 102. Sensors on the ultrasound device 120 used to determine a position of the ultrasound device 120 relative to a reference position at the LINAC 102 may be used to register data generated by the ultrasound device 120 with the LINAC 102. Any number of other registration methods may be used.

The controller 122 may compare the ultrasound data generated by the ultrasound system 400 and the three-dimensional representation of the treatment volume 106 in the treatment plan 124 stored in the memory 126. The controller 122 may determine differences (e.g., translation and/or rotation) between the generated ultrasound data and the stored three-dimensional representation of the treatment volume 106 in the treatment plan 124. Based on the generated ultrasound data and/or the determined differences, the controller 122 may control the LINAC 102, the scanning magnets 110, and the beam shaping device 112, such that radiation reaching the region outside of the target volume 106 is minimized. The controller 122 may control more or fewer components to minimize the radiation reaching the region outside of the target volume 106.

The treatment region, as represented in the ultrasound data, may be determined by correlation. For example, the ultrasound data is filtered to enhance edges. The edges of the tumor, as represented in the ultrasound data, may be enhanced. The treatment region or shape is translated, rotated, and/or scaled relative to the three-dimensional representation of the target volume 106 in the treatment plan 124. The translation, rotation, and/or scaling with a greatest similarity provides the current position of the treatment region relative to the ultrasound device 120. Using the transform to the LINAC system, the position of the treatment region within the patient relative to the LINAC system is determined.

Rather than correlation of ultrasound data with a treatment plan or representation of the spatial distribution of the target, the current position of the target may be determined from the ultrasound data. The user may outline the tumor in the current ultrasound data. A processor may track the target volume based on the user indication. Alternatively, the processor detects the target volume based on any image processing. For each time, the target volume is detected again or is tracked from a prior detection. The transform of the ultrasound coordinate system to the therapy device coordinate system relates the position of the target volume relative to the ultrasound space to the therapy space.

In one embodiment, if the target volume 106 moves out of the field of view of the LINAC 102 (e.g., as determined by the generated ultrasound data) while the LINAC 102 is irradiating the target volume 106, the controller 122 may turn off the LINAC 102, such that the particle beam 104 is not delivered to the target object 108 (e.g., active gating). This may prevent the LINAC 102 from delivering radiation to the target volume 106 when the patient accidentally moves (e.g., sneezes). When the target volume 106 is repositioned into the field of view of the LINAC 102, the controller 122 may turn on the LINAC 102, such that the particle beam 104 is again delivered to the target object 108 (e.g., the target volume 106).

Additionally or alternatively, when the target volume is within the field of view of the LINAC 102, the controller 122 may control the scanning magnets 110 and/or the beam shaping device 112 to move and/or shape the particle beam 104 to correct for motion of the target volume 106, as determined by the ultrasound device 120. The controller 122 may control the scanning magnets 110 and/or the beam shaping device 112 based on the determined differences between the generated ultrasound data and the stored three-dimensional representation of the treatment volume 106 in the treatment plan 124 and/or the generated ultrasound data. For example, as the target volume 106 moves while the patient is breathing, the controller 122 may control the individual leaves of the beam shaping device 112 to approximately match the shape of the target volume 106 (e.g., a current shape of the target volume 106) based on the determined differences between the generated ultrasound data and the stored three-dimensional representation of the treatment volume 106 in the treatment plan 124 and/or the generated ultrasound data. The controller 122 may control the scanning magnets 110 to move the particle beam 104 to track the motion of the target volume 106 based on the determined differences between the generated ultrasound data and the stored three-dimensional representation of the treatment volume 106 in the treatment plan 124 and/or the generated ultrasound data.

In one embodiment, the controller 122 may change the treatment plan 124 based on the determined differences between the generated ultrasound data and the stored three-dimensional representation of the treatment volume 106 in the treatment plan 124 and/or the generated ultrasound data. For example, the controller 122 may change the treatment plan 124 by changing the sequence of delivery segments, the beam shape (i.e., the shape and/or the orientation of the beam shaping device 112), the beam dose, the beam energy, and/or the gantry angles defining the range or span of the segment (e.g., the upper limit and the lower limit), within which the radiation dose is to be delivered, defined in the treatment plan 124. The treatment plan 124 may be changed in combination with the other methods of control discussed above. The treatment plan 124 is altered to account for the change in position of the target.

The particle therapy system 100 may include any number of other components. For example, the particle therapy system 100 may include detectors for monitoring beam parameters and/or a detection device operable to record motion of the target object 108, for example.

The LINAC 102 and the ultrasound device 120 may not interfere with each other. In other words, no influence on the energy disposition of secondary electrons is expected. The LINAC 102 may be specified and produced as an independent system component; thus, the interaction between the LINAC 102 and the ultrasound device 120 may be minimized. No ionizing radiation is used for imaging during organ tracking, and compared to computed tomography (CT), soft tissue contrast and organ segmentation information may be improved. The combination of the LINAC 102 and the ultrasound device 120 may also be less expensive than CT-based system and magnetic resonance imaging (MRI)-based systems.

FIG. 5 shows a flowchart of one embodiment of a method of correcting for target volume motion for an irradiation of a target volume of a patient with a particle beam generated by a radiotherapy device. The method may be performed using the particle therapy system 100 shown in FIG. 1 or another image-guided therapy system. The method is implemented in the order shown, but other orders may be used. Additional, different, or fewer acts may be provided. Similar methods may be used for correcting for target volume motion for an irradiation of a target volume of a patient.

In act 500, a support structure is positioned on or adjacent to an external surface (e.g., an external surface of the skin) of a target object (e.g., a patient). The support structure may support at least one ultrasound transducer. The support structure may be positioned outside of a beam path or all beam paths of the particle beams generated by the radiotherapy device for a given treatment. The beam path may extend from the radiotherapy device to the target volume to be irradiated (e.g., treated). The support structure may be positioned such that a distance between the transducer and the target volume is minimized, while keeping absorbing materials out of the beam path. The support structure may be sized to be wrapped around a part (e.g., an arm, a leg, the chest) of the patient. The support structure may include a tightening mechanism and/or a locking mechanism, such that the support structure and thus the at least one transducer are generally fixed positionally relative to the external surface of the patient. An imaging device such as, for example, an MRI device, a CT device, another X-ray device, or any other now known or later discovered imaging devices may be used to aid in the positioning of the support structure relative to the target volume and/or the radiotherapy device.

In one embodiment, two or more support structures are positioned on opposite sides of a target region above (e.g., an area on the external surface representing the target volume projected to the external surface in a direction parallel to the particle beam) the target volume. The two support structures may each support a plurality of transducers along the length of the support structure (i.e., around the circumference of the body part of the patient when the two support structures are wrapped around the body part). In another embodiment, the support structure may support a plurality of transducers (e.g., four transducer arrays) and may include an opening. The plurality of transducers may be disposed around the opening in the support structure. The support structure may be positioned on the external surface of the patient, such that a center of the opening aligns with a center of the target volume, and the plurality of transducers are disposed around the target region and thus the target volume.

In act 502, image data representing at least part of the target volume is generated using the ultrasound transducer. In order to image the target volume (e.g., cancerous tissue) and/or a region outside of the target volume (e.g., healthy tissue), at least one ultrasound beam generated by the transducer may be directed towards a center of an exposure region (e.g., the target volume) of the particle beam or the outside region. Additionally or alternatively, the ultrasound beams sweep tissue regions adjacent to and/or including the exposure region. The at least one ultrasound transducer may be operated at a frame rate of, for example, 10 or more frames per second to provide a temporal resolution sufficient to stabilize the particle beam in the patient. Other frame rates, however, may be used. Multiple ultrasound transducers may increase volume coverage and temporal resolution.

In act 504, the target volume is irradiated with the radiotherapy device. The radiotherapy device may accelerate the particle beam to energies between 4 and 25 MeV, for example, before being directed to the target volume. A sequence of delivery segments, within which discrete points are described by, for example, the shape of the particle beam (i.e., a shape of the collimator used to shape the particle beam), positions of the particle beam, a beam dose, and/or other specifications may be set in a predetermined treatment plan. The predetermined treatment plan may also include a three-dimensional representation of the target volume. The three-dimensional representation may be generated by an imaging device prior to the treatment. The predetermined treatment plan may, for example, be stored in and referenced from a memory.

The radiotherapy device may irradiate the target volume using an IMRT methodology. The IMRT methodology may be a stop-and-shoot IMRT methodology, where a gantry of the radiotherapy device rotates and stops at one or more gantry angles, at which the radiotherapy device delivers radiation to the target volume. Alternatively, the radiotherapy device may deliver radiation to the target volume while the gantry of the radiotherapy device is rotating. The radiotherapy device may deliver radiation to the target volume continuously during rotation of the gantry, or may deliver radiation to the target volume in segments (e.g., between gantry angles 15 degrees to 30 degrees and 45 degrees to 60 degrees) of the rotation of the gantry. The type and number of structural supports and the number of transducers may be determined based on which IMRT methodology is used. For example, if the radiotherapy device delivers radiation to the target volume while the gantry of the radiotherapy device rotates, the two structural supports each supporting a plurality of transducers along the length of the structural support may be used, such that the target volume may be imaged in line with each angle of incidence around the gantry.

In one embodiment, the plurality of transducers may include at least one high-intensity focused ultrasound (HIFU) transducer operable to emit an HIFU beam. The HIFU transducer may be guided by other ultrasound transducers of the plurality of ultrasound transducers. The HIFU transducer may be used to ablate the treatment volume.

During irradiation of the treatment volume, the patient may move. For example, while the patient lies on a treatment table or bed, the patient breathes. The target volume (e.g., a tumor) may move with the motion of the chest of the patient during breathing. Thus, the target volume may be outside of a treatment position (e.g., a position of the target volume, at which the target volume is irradiated with the particle beam). Irradiation with the target volume out of position as defined by the treatment plan (e.g., the three-dimensional representation of the target volume) at any time during the medical treatment procedure may result in improper application of the radiation. Without corrective measures, healthy tissue of the patient (e.g., a region outside of the target volume) may be irradiated with the particle beam.

In act 506, the irradiation of the target volume is controlled based on a comparison of the generated image data to a predetermined treatment plan to correct for motion of the target volume during the irradiation. The comparison occurs before irradiation, during irradiation, and/or after irradiation. The comparison may be between the three-dimensional representation of the treatment volume of the treatment plan and the generated image data. The three-dimensional representation of the treatment volume presumes a particular position of the target volume relative to the radiotherapy device. To find an offset from this arrangement, the three-dimensional representation of the treatment volume and the generated image data may be registered to each other, to a coordinate system of the radiotherapy device, or another coordinate system. The comparison may include calculating a difference (e.g., a translational difference, scale difference, and/or a rotational difference) between the three-dimensional representation of the treatment volume and the generated image data.

In one embodiment, the controlling may include moving the particle beam and/or shaping the particle beam based on the calculated difference to track the movement and/or change in shape of the target volume relative to the radiotherapy device. The particle beam may be moved using scanning magnets of the radiotherapy device. The particle beam may be shaped using a beam shaping device (e.g., a multileaf collimator). In another embodiment, the controlling may include turning the particle beam on and off based on a position of the target volume relative to a field of view of the radiotherapy device (e.g., as determined by the generated image data and/or the calculated difference). For example, when the target volume moves outside of the field of view (e.g., wholly or partially) of the radiotherapy device due to breathing by the patient, the controlling may include turning the particle beam off. When the target volume moves back into the field of view of the radiotherapy device, the controlling may include turning the particle beam back on. In one embodiment, the controlling includes turning the particle beam off when the target volume moves outside of the field of view of the radiotherapy device, and turning the particle beam on and moving the particle beam to track the movement of the target volume when the target volume is within the field of view of the radiotherapy device. In one embodiment, the controlling may include changing the treatment plan based on the calculated difference between the generated image data and the three-dimensional representation of the treatment volume and/or the generated image data. For example, the treatment plan may be changed by, for example, changing the sequence of delivery segments, the beam shape, the beam dose, the beam energy, and/or the gantry angles defining the range or span of the segment, within which the radiation dose is to be delivered, to compensate for the motion of the target volume.

In one embodiment, the generated image data may be used to determine where the radiation dose went within the target object to generate a dose record. The dose record may be used to evaluate therapy response and/or to re-plan further treatments and/or for a hospital's quality assurance.

While the present invention has been described above by reference to various embodiments, it should be understood that many changes and modifications can be made to the described embodiments. It is therefore intended that the foregoing description be regarded as illustrative rather than limiting, and that it be understood that all equivalents and/or combinations of embodiments are intended to be included in this description.

1. A system for irradiating a target volume with a radiation or particle beam, the system comprising:
a radiotherapy device operable to irradiate the target volume with the radiation or particle beam;
an ultrasound device comprising an ultrasound transducer, the ultrasound device being operable to generate ultrasound data representing at least part of the target volume, the ultrasound transducer being positioned adjacent to the target volume;
a memory configured to store a treatment plan for irradiating the target volume with the radiation or particle beam; and
a processor operatively connected to the radiotherapy device, the ultrasound transducer, and the memory, the processor being configured to:
generate image data corresponding to the at least part of the target volume based on the generated ultrasound data; and
control the radiotherapy device based on the generated image data and the stored treatment plan.

2. The system of claim 1, wherein the radiotherapy device is a linear accelerator (LINAC), a Cobalt-based radiation therapy or radiation surgery system, or a particle therapy system.

3. The system of claim 1, wherein the ultrasound transducer is a capacitive micromachined ultrasonic transducer (CMUT), a piezoelectric transducer, a composite-based transducer, or a combination thereof.

4. The system of claim 1, wherein the target volume is a tumor in a patient, and
wherein the ultrasound transducer is positioned on or adjacent to an external surface of the patient, outside of a path of the radiation or particle beam.

5. The system of claim 4, wherein the ultrasound device comprises a support structure, the support structure being positionable on or adjacent to the external surface of the patient, and
wherein the ultrasound transducer is supported by the support structure.

6. The system of claim 5, wherein the ultrasound device comprises a plurality of ultrasound transducers, the plurality of ultrasound transducers including the ultrasound transducer and being supported by the support structure, the support structure being disposed at least partially around part of the patient.

7. The system of claim 6, wherein the plurality of ultrasound transducers are positioned on the support structure and the support structure is positionable on the patient, such that the plurality of ultrasound transducers at least partially surround the treatment beam when the treatment beam irradiates the target volume with the radiation or particle beam.

8. The system of claim 6, wherein the plurality of ultrasound transducers is a first plurality of ultrasound transducers, and the support structure is a first support structure,
wherein the system further comprises:
a second support structure, the second support structure being disposed at least partially around the part or another part of the patient; and
a second plurality of ultrasound transducers, the second plurality of ultrasound transducers being supported by the second support structure, and
wherein the first support structure and the second support structure are positioned on or adjacent to the external surface of the patient on opposite sides of the treatment beam.

9. The system of claim 8, wherein the first support structure is a first belt that supports the first plurality of ultrasound transducers, and the second support structure is a second belt that supports the second plurality of ultrasound transducers.

10. A method of correcting for target volume motion for an irradiation of a target volume of a patient with a radiation or particle beam, the method comprising:
positioning a support structure on or adjacent to an external surface of the patient, the support structure supporting an ultrasound transducer, the ultrasound transducer being outside of a beam path of the radiation or particle beam from a radiotherapy device to the target volume;
generating image data representing at least part of the target volume using the ultrasound transducer;
irradiating the target volume with the radiotherapy device;
controlling the irradiation of the target volume based on the generated image data.

11. The method of claim 10, wherein the controlling comprises turning the radiation or particle beam on and off based on a comparison of the generated image data and a predetermined treatment plan.

12. The method of claim 10, wherein the controlling comprises adjusting a predetermined treatment plan based on a comparison of the generated image data and the predetermined treatment plan.

13. The method of claim 10, wherein the controlling comprises shaping, steering, or shaping and steering the radiation or particle beam based on the generated image data.

14. The method of claim 10, wherein the ultrasound device is a first ultrasound device of a plurality of ultrasound devices supported by the support structure.

15. In a non-transitory computer-readable storage medium that stores instructions executable by one or more processors to correct for motion of a target volume during a radiation therapy with a radiation or particle beam, the instructions comprising:
receiving data representing at least part of the target volume from an ultrasound transducer, the ultrasound transducer being outside of a beam path of the radiation or particle beam from a radiotherapy device to the target volume; and
controlling irradiation of the target volume, the controlling comprising turning the radiation or particle beam on and off, moving the radiation or particle beam, changing a shape of the radiation or particle beam, or a combination thereof based on the data.

16. The non-transitory computer-readable storage medium of claim 15, further comprising:
receiving a predetermined treatment plan, wherein the predetermined treatment plan comprises three-dimensional (3D) image data representing the target volume, data representing one or more gantry angles of the radiotherapy device, at which the radiotherapy device irradiates the target volume, one or more shapes of a collimator of the radiotherapy device at the one or more gantry angles, the collimator shaping the radiation or particle beam, one or more intensities of the radiation or particle beam, or a combination thereof; and
comparing the received data to the received treatment plan;
wherein controlling comprises controlling based on the comparing.

17. The non-transitory computer-readable storage medium of claim 15, wherein receiving data representing at least part of the target volume comprises receiving three-dimensional (3D) image data representing at least the part of the target volume from the ultrasound transducer.

18. The non-transitory computer-readable storage medium of claim 15, wherein receiving data representing at least part of the target volume from the ultrasound transducer comprises receiving data representing at least part of the target volume at a plurality of time points.

19. The non-transitory computer-readable storage medium of claim 18, wherein the controlling comprises turning the radiation or particle beam off when the target volume is outside of a field of view of the radiotherapy device and turning the radiation or particle beam on when the target volume is inside the field of view of the radiotherapy device.

20. The non-transitory computer-readable storage medium of claim 19, wherein the controlling comprises moving the particle beam to track the target volume when the target volume is inside of the field of view of the radiation therapy device.
